(19) United States
(12) Patent Application Publication
(10) Pub. No.: US 2020/0347354 A1
(43) Pub. Date: Nov. 5, 2020

(54) METHOD OF ISOLATION OF STEM CELLS FROM HYPERTHERMIA CONDITIONED TISSUES AND USING THE SAME

(71) Applicant: BAYLX, INC., Irvine, CA (US)

(72) Inventors: Wenbin LIAO, Irvine, CA (US); Yongjun LIU, Irvine, CA (US)

(21) Appl. No.: 16/962,501

(22) PCT Filed: Feb. 8, 2019
(86) PCT No.: PCT/US2019/017196

§ 371 Date: Jul. 15, 2020

Related U.S. Application Data
(60) Provisional application No. 62/632,988, filed on Feb. 20, 2018.

(51) Int. Cl. C12N 5/0775; A61K 35/51

(52) U.S. Cl. CPC C12N 5/0665; C12N 2523/00; A61K 35/51

(57) ABSTRACT

The invention provides methods to obtain stem cells and the use of such stem cells. Provided methods are isolation of stem cells from hyperthermia-treated stem cell-containing tissues. Practicing these methods will substantially increase the yield of stem cell isolation, the proliferation rates of the stem cells in subculture, and the therapeutic potencies of the stem cells for their therapeutic use in diseases including, but not limited to, autoimmune diseases, liver diseases and cancer.

This application claims the benefit of U.S. Provisional Application No. 62/632,988, filed on Feb. 20, 2018, which is incorporated by reference for all purposes as if fully set forth herein.

### TECHNICAL FIELD

The present disclosure relates to method using hyperthermic conditions to manipulate mesenchymal stem cell (MSC)-containing tissues or mesenchymal stem cells to obtain MSC with enhanced proliferation capacity and therapeutic potencies.

### BACKGROUND

A major challenge in the field of mesenchymal stem cells (MSC)-based therapy is to manufacture enough amount of cells at an appropriate stage without the need of extensive passaging in vitro. MSCs have a limited life span and during in vitro expansion, the proliferation rate of MSC will slow down. Also, the therapeutic potencies of MSC can diminish after the in vitro culture expansion.

### SUMMARY

MSC based therapy could benefit from methods to maintain or enhance the therapeutic potencies of MSCs, including, but not limited to, anti-inflammation, immunomodulation, secretion of protein, migration and differentiation of the stem cell in order to achieve more effective and consistent results in the treatment of diseases.

MSC are usually quiescent in physiological conditions, while in pathological environments such as an infection or injury, they are activated and functional to repair or regenerate the damaged tissues by secreting trophic or anti-inflammatory factors and/or replacing the lost cells through differentiation. Therefore, according to several embodiments disclosed herein, preconditioning of MSC with disease-mimicking environments entitle the MSC with more powerful proliferation and therapeutic functions. For example, hypoxia has been introduced into MSC culture to control their replication and differentiation potential [Patent PCT/US2010/032457, “Compositions of stem cells and stem cell factors and methods for their use and manufacture”]. In a distinct approach discussed in more detail below, there are provided methods using hyperthermic conditions to manipulate MSC-containing tissues (or MSC themselves directly) to obtain MSC with enhanced proliferation capacity and therapeutic potencies.

In several embodiments, there are provided methods employing hyperthermic conditions to treat MSC-containing human or other mammalian tissues to activate the MSC in situ, and as a consequence, to substantially increase the proliferation rate and yield of the stem cell isolation, and/or to increase the therapeutic potencies including anti-inflammation, immunomodulation, secretion of protein, migration and differentiation of the MSC after isolation. In particular, in several embodiments, provided are methods for deriving activated/primed MSC from hyperthermia-treated human umbilical cord tissues. In several embodiments, the human umbilical cord tissues are from full-term deliveries. In additional embodiments, provided are methods using hyperthermia to treat both MSC-containing tissues (before isolation) and MSC (after isolation) to increase the proliferation rate and therapeutic potencies including anti-inflammation, immunomodulation, secretion of protein, migration, differentiation of the MSC. Also provided are compositions and use of the stem cells, including but not limited to MSC, neural stem cells and hematopoietic stem cells, derived from the methods disclosed herein as therapies for, including but not limited to, autoimmune diseases, neural diseases, liver diseases or cancer.

In several embodiments, a method of treating mesenchymal stem cells (MSC)-containing tissues with hyperthermic conditions in order to isolate MSC's with an enhanced proliferation rate when compared to a control is presented. In several embodiments, the method comprises: isolating MSC-containing tissue, culturing the MSC containing tissue in an optimized medium, exposing the tissue to hyperthermic conditions, and providing therapy to a patient suffering from a medical disease.

In several embodiments, the hyperthermic conditions produce MSC-containing tissue with an enhanced proliferation rate and therapeutic potencies when compared to a control.

In several embodiments, the hyperthermic conditions comprise of temperatures ranging, and including, between 37 to about 40 degrees Celsius (° C.). Higher temperatures are also used, in several embodiments.

In several embodiments, the optimized medium used to culture the MSC containing tissue includes one or more supplements selected from growth factors (bFGF, EGF) and hormones (Dexamethasone), antibiotics (Pen/Strap, Amphotericin B), serum, and xeno-free serum replacement.

In several embodiments, the MSC-containing tissue may come from umbilical cord. The human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

In one embodiment, the human umbilical cord tissues are disaggregated by cutting the tissue without enzymatic digestion before culturing in the optimized medium.

In one embodiment the human umbilical cord tissues are cut into small sections comprising 1-1.5 mm in diameter and not washed in order to preserve the environment of the tissues after injury by cutting or agitation and then directly used for culturing.

In several embodiments, the MSC containing tissues may be taken from bone marrow, adipose tissues, blood, amniotic fluid, dental pulp and placenta.

In several embodiments, the MSC with an enhanced proliferation rate and therapeutic potencies are suitable use to treat a medical disease including: anti-inflammation, immunomodulation, secretion of protein, migration differentiation of the MSC.

In several embodiments, the hyperthermic conditions may include one or more of the followings in addition to hyperthermia treatment: cutting or agitation; damage associated molecular patterns (DAMPs) such as HGMB1, S100B; pathogen associated molecular patterns (PAMPs) such as peptidoglycan (PGN), and double-stranded RNA; heat shock proteins; inflammatory cytokine (such as TNF-α, IFN-γ, interleukin-1 (IL-1) etc.) stimulation; stem cell migration related proteins (CXCL12, G-CSF, SCF, PDGF-BB, etc.) stimulation and hypoxia (<20% O2).

In one embodiment, the damage associated molecular patterns (DAMPs) include, but are not limited to, HGMB1 (the chromatin-associated protein high-mobility group box 1), S100B, Purine metabolites (ATP, adenosine, and uric acid).

In one embodiment the pathogen associated molecular patterns (PAMPs) include, but are not limited to, peptidoglycan (PGN), and double-stranded RNA.

In one embodiment the heat shock proteins include, but are not limited to, HSP 40, HSP 60, HSP 70, HSP 72, HSP 78, HSP 90, HSP 104, HSP 110.

In one embodiment the inflammatory cytokines include, but are not limited to, TNF-α, IFN-γ, TGF-β, IL-1, IL-6, IL-8.

In one embodiment the stem cell migration related proteins include, but are not limited to, CXCL12 (stromal cell-derived factor-1: SDF-1), CCL2 (monocyte chemotactic protein-1: MCP-1), CCL5 (regulated on activation, normal T cell expressed and secreted: RANTES), CCL8, G-CSF, SCF, PDGF-BB.

In several embodiments, the therapy may be used to treat autoimmune diseases or liver diseases, or cancer.

In several embodiments, the therapy may treat various autoimmune diseases. These diseases may comprise Crohns' diseases, ulcerative colitis, multiple sclerosis, rheumatoid arthritis, systemic lupus erythematosus, autoimmune pancreatitis, Type 1 diabetes, systemic sclerosis.

In several embodiments, the therapy may treat various liver diseases. These liver diseases may comprise liver failure, cirrhosis, hepatic steatosis, hepatitis.

A method of treating mesenchymal stem cells (MSC)-containing tissues with hyperthermic conditions for the isolation of the MSC with enhanced proliferation rate and therapeutic potencies compared to the control MSC isolated without hyperthermic conditions.

In alternative embodiments, the hyperthermic conditions are also used to treat MSC after isolation, or treat both MSC-containing tissues and MSC to produce MSC with enhanced proliferation rate and therapeutic potencies.

The hyperthermic conditions comprise at least hyperthermia which is “temperatures that are higher than human normal physiologic core body temperature 37 degrees Celsius (° C.)”. Hyperthermic conditions used in this invention are, including but not limited to, from 37 to 40 degrees Celsius.

The MSC-containing tissues comprise preferably human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

Other examples of the MSC-containing tissues include, but are not limited to, bone marrow, adipose tissues, blood, amniotic fluid, dental pulp and placenta.

The therapeutic potencies of the MSCs include, but are not limited to, anti-inflammation, immunomodulation, secretion of protein, migration differentiation of the MSC.

In other embodiments, the hyperthermic conditions may include one or more of the followings in addition to hyperthermia treatment: mechanical cutting or agitation; damage associated molecular patterns (DAMPs) such as HGMB1, S100B; pathogen associated molecular patterns (PAMPs) such as peptidoglycan (PGN), and double-stranded RNA; heat shock proteins; inflammatory cytokine (such as TNF-α, IFN-γ, interleukin-1 (IL-1) etc.) stimulation; stem cell migration related proteins (CXCL12, G-CSF, SCF, PDGF-BB, etc.) stimulation and hypoxia (<20% 02).

The damage associated molecular patterns (DAMPs) include, but are not limited to, HGMB1 (the chromatin-associated protein high-mobility group box 1), S100B, Purine metabolites (ATP, adenosine, and uric acid).

The pathogen associated molecular patterns (PAMPs) include, but are not limited to, peptidoglycan (PGN), and double-stranded RNA.

The heat shock proteins include, but are not limited to, HSP 40, HSP 60, HSP 70, HSP 72, HSP 78, HSP 90, HSP 104, HSP 110.

The inflammatory cytokines include, but are not limited to, TNF-α, IFN-γ, TGF-β, IL-1, IL-6, IL-8.

The stem cell migration related proteins include, but are not limited to, CXCL12 (stromal cell-derived factor-1: SDF-1), CCL2 (monocyte chemotactic protein-1: MCP-1), CCL5 (regulated on activation, normal T cell expressed and secreted: RANTES), CCL8, G-CSF, SCF, PDGF-BB.

The method for the isolation of MSC from human umbilical cord tissues in claim 3 wherein tissue disaggregation is achieved preferably by mechanical cutting without enzymatic digestion.

The methods include wherein the human umbilical cord tissues are mechanically cut into small sections (1-1.5 mm in diameter) and directly used for tissue culture, in which washing of the sections are NOT used in order to preserve the environments after mechanical injury by cutting or agitation.

Methods of culture and expansion of MSC with optimized medium and supplements such as growth factors (bFGF, EGF) and hormones (Dexamethasone), antibiotics (Pen/Strap, Amphotericin B), Serum-containing or free; xeno-containing or free

Compositions and use of MSC for the treatment of autoimmune diseases or liver diseases and cancer are provided as well, according to several embodiments.

The methods of treating autoimmune diseases include, but are not limited to, Crohns' diseases, ulcerative colitis, multiple sclerosis, rheumatoid arthritis, systemic lupus erythematosus, autoimmune pancreatitis, Type 1 diabetes, systemic sclerosis.

The methods of treating liver diseases include, but are not limited to, liver failure, cirrhosis, hepatic steatosis, hepatitis.

### BRIEF DESCRIPTION OF THE DRAWINGS

FIGS. 1A-1B shows the total numbers of mesenchymal stem cells (MSC) that can be isolated from each group in FBS system. Provided are (A) the table and (B) the graph indicating the number of MSC was higher “crawling” from the tissue blocks induced at 38° C. for 1 hour. Each block on average produced about 20% more MSC than that in the 37° C. group.

FIGS. 2A-2E shows the MSC isolated from each group under hyperthermic conditions in FBS system. Provided are representative pictures of cells isolated at (A) 37° C.; (B) 38° C., 1 hr; (C) 38° C., 30 min; (D) 39° C., 30 min and (E) 39° C., 1 hr.

FIGS. 3A-3B shows the total numbers of MSC that can be isolated from each group in UG system. Provided are (A) the table and (B) the graph indicating the numbers of MSC were higher “crawling” from the tissue blocks hyperthermically induced at 38° C. or 39° C. for 1 hour. Each block produced 40-80% more MSC than that in the 37° C. group.

FIGS. 4A-4E shows the MSC isolated from each group under hyperthermic conditions in UG system. Provided are representative pictures of cells isolated at (A) 37° C.; (B) 38° C., 1 hr; (C) 38° C., 30 min; (D) 39° C., 30 min and (E) 39° C., 1 hr in FBS system.

FIGS. 5A-5E shows the morphology of MSC isolated from each group under hyperthermic conditions in UG system. Provided are representative pictures of cells isolated at (A) 37° C.; (B) 38° C., 1 hr; (C) 38° C., 30 min; (D) 39° C., 30 min and (E) 39° C., 1 hr in UG system.

FIG. 6 shows the cells of 38° C./1 hr group in FBS system increased most rapidly along with passaging. Provided are total numbers of MSC of different passages isolated under each hyperthermic condition in FBS system.

FIG. 7 shows the cells of 38° C./1 hr group in UG system increased most rapidly along with passaging. Provided are total numbers of MSC of different passages isolated under each hyperthermic condition in FBS system.

FIGS. 8A-8B shows the total numbers of cells isolated in each group in P6 in (A) FBS or (B) UG system.

FIG. 9 shows that cell diameters increase along with the passaging in FBS system. Provided is a graph showing the cell diameters of MSC of each passage isolated under different hyperthermic conditions in FBS system.

FIG. 10 shows that cell diameters increase along with the passaging in UG system. Provided is a graph showing the cell diameters of MSC of each passage isolated under different hyperthermic conditions in UG system.

FIG. 11 shows that cells isolated hyperthermically present adequate MSC phenotypes. Provided are flow cytometric results (using biomarkers CD19, CD34, CD11b, CD45 and HLA-DR (negative) as well as CD73, CD90 and CD105 (positive)) of MSC isolated in different groups.

FIGS. 12A-12D shows that MSC of all group presented good proliferation rates. Provided are graphs of proliferation rates (CCK-8 cell proliferation assay) of MSC isolated under different hyperthermic conditions in (A and B) FBS or (C and D) UG system.

FIGS. 13A-13E shows that the senescence of MSC from 39° C. inducing groups was significantly higher than that from the other groups in FBS system. Provided are representative pictures of SA-β-gal staining for MSC isolated at (A) 37° C.; (B) 38° C., 1 hr; (C) 38° C., 30 min; (D) 39° C., 30 min and (E) 39° C., 1 hr in FBS system.

FIGS. 14A-14E shows that the senescence of MSC from 39° C. inducing groups was significantly higher than that from the other groups in UG system. Provided are representative pictures of SA-β-gal staining for MSC isolated at (A) 37° C.; (B) 38° C., 1 hr; (C) 38° C., 30 min; (D) 39° C., 30 min and (E) 39° C., 1 hr in UG system.

FIGS. 15A-15B shows that MSC isolated under each hyperthermic condition can significantly inhibit the secretion of TNF-α from PBMC. Provided are graphs indicating the immunosuppressive effects from (A) MSC-FBS or (B) UG-FBS on TNF-α secretion of PBMC.

### DETAILED DESCRIPTION

Several embodiments relate to the use of hyperthermia-like conditions in the presence or absence of other disease-mimicking conditions to treat mesenchymal stem cell (MSC)-containing tissues or stem cells directly to produce MSC having enhanced proliferation and therapeutic potencies including anti-inflammation, immunomodulation, secretion of protein, migration, homing, engraftment or differentiation characteristics. In very general terms, such embodiments may be practiced by incubating the stem cell-containing tissues in hyperthermic conditions with or without other disease-mimicking conditions, isolating a population of stem cells from the hyperthermia conditioned tissues, culturing the population of stem cells in vitro with or without exposing to hyperthermia in the presence or absence of other disease-mimicking conditions the stem cell population to produce a population of stem cells having at least enhanced proliferation and/or therapeutic characteristics. Methods of using the presently disclosed stem cells are also contemplated as embodiments of the invention.

Hyperthermia

The phrase “hyperthemia”, “hyperthemic conditions” and “high temperature” shall be given their ordinary meaning and shall refer to “temperatures that are higher than normal physiologic human body temperature 37 degrees Celsius”. Hyperthermic conditions generally mean any temperatures that are higher than 37 degrees Celsius, preferably 37 to 41 degrees Celsius, more preferably 38 degrees Celsius. It is understood that normal physiologic body temperatures vary in different tissues or organs, thus, in some embodiments, the conditions employed for stem cell containing-tissues will depend on the tissues and organ origins; such conditions are known to the skilled artisan. Physiologic body temperatures are the range of oxygen levels normally found in healthy tissues or organs.

Disease-mimicking conditions for use with the invention include, but are not limited to, pro-inflammatory cytokines, mechanical cutting or agitation, damage associated molecular patterns (DAMPs), pathogen associated molecular patterns (PAMPs), heat shock proteins, stem cell migration related proteins and combinations thereof.

In several embodiment, the hyperthermic conditions comprise a temperature between about 37 to 41 degrees Celsius. In additional embodiments, hyperthermic conditions comprise a temperature between about 37.5 to about 38.5 degrees Celsius. In further embodiment, hyperthermic conditions comprise a temperature between about 38 degrees Celsius. In several embodiments, the hyperthermic conditions fall in any of these ranges generally or hyperthermic conditions between any of these ranges that mimics physiological hyperthermic conditions for the particular cells. Thus, according to several embodiments, the temperature culture conditions are set at 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 41.0 degrees Celsius or any other temperate conditions between any of these figures.

In one embodiment, the hyperthermic conditions are introduced during the production of the stem cells. One skilled in the art will appreciate that the timing of the introduction of hyperthermic conditions for the stem cell production will depend on the stem cell characteristics that are desired. Hyperthermic conditions may be introduced at any time during the production of the stem cells. Hyperthermic conditions are introduced at times including, but not limited to, after collection of the stem cell-containing tissues, after the disaggregation of such tissue samples into any sizes of segments by mechanical or enzymatic methods, during the primary culture (e.g. explant) of stem cells, during the in vitro expansion of the stem cells (e.g. over multiple cell passages), during priming (e.g. when stem cells are induced to assume a desired biological activity prior to injection into a subject), and combinations thereof.

MSC-containing tissues and/or MSC can be exposed to hyperthermic conditions under any methodologies that permit the production of stem cells with an enhanced proliferation rate and therapeutic potencies including anti-inflammation, immunomodulation, secretion of protein, migration, homing, engraftment or differentiation characteristics as disclosed herein. “Enhanced,” when used to refer to a stem cell's proliferation rate, means any measurable increases in the stem cell's mitotic cell division rate. When used to refer to a stem cell's therapeutic potencies, “enhanced” means retaining, or inhibiting the loss of, a stem cell's anti-inflammation, immunomodulation, secretion of protein, migration, homing, engraftment or differentiation characteristics as the stem cell is expanded and passaged in in vitro culture.

Physiologic or hyperthermic culturing conditions can be maintained by using commercially available chambers in which the range of hyperthermic conditions as described herein can be reached. Hyperthermic culturing conditions can also be achieved by incubating the stem cell-containing tissues and/or stem cells in culture medium pre-warmed to the temperature of hyperthermic conditions and maintained by contacting the bottom of the culture to a temperature-controlled heating platform. In certain embodiments, any two or all of the methods: temperature-controlled chamber, pre-warmed incubating medium and temperature-controlled heating platform as described herein, are used to achieve hyperthermic conditions.

In one embodiment, the length of time when the MSC-containing tissues and/or stem cells are exposed to hyperthermic conditions is controlled. According to such embodiments, stem cell-containing tissues and/or stem cells may be exposed to hyperthermic conditions for any amount of time that enhances the proliferation and/or therapeutic potencies of the stem cells as disclosed herein. This may be 5 minutes, 10 minutes, 15 minutes, 20 minutes, 30 minutes, 45 minutes, 60 minutes or more, or this time may be continuous (e.g. the entire time that from the tissue collection to the harvest of stem cells cultured in vitro). The typical incubation conditions for the culture is 5% CO₂, 20% O₂ with 100% humidity and the temperature is about 37 degrees Celsius if the culture is not in the timeframe for hyperthermic exposure.

Mesenchymal Stem Cell-Containing Tissues and Mesenchymal Stem Cells

In several embodiments practiced with mesenchymal stem cell (MSC)-containing tissues and MSC, the invention may be used to manipulate any tissues where stem cells reside and any stem cells (or combination of stem cells) that are capable of being enhanced under the methods of this invention. Suitable stem cell-containing tissues for use with the invention include, but are not limited to, human or other mammalian umbilical cord tissues, umbilical cord blood, bone marrow aspirate, adipose tissues (i.e. fat), placenta, amniotic fluid, dental pulp, peripheral blood, bone, cartilage, heart, lung, kidney, liver, brain, spinal cord, skin and tumor tissues. Suitable stem cell for use with the invention include, but are not limited to, pluripotent embryonic stem cells, MSC, neural stem cells, hematopoietic stem cells, endothelial progenitor cells, liver stem cells, heart stem cells, skin stem cells and combinations thereof.

In some of the embodiments, MSC-containing tissues are used. That is, using hyperthermia in the presence or absence of other disease-mimicking conditions to treat MSC-containing tissues produce MSC having enhanced proliferation and therapeutic potencies including anti-inflammation, immunomodulation, secretion of protein, migration, homing, engraftment or differentiation characteristics. MSC can be isolated any human and other mammalian tissues or organs, thus MSC-containing tissues include, but are not limited to, human or other mammalian umbilical cord tissues, umbilical cord blood, bone marrow aspirate, adipose tissues (i.e. fat), placenta, amniotic fluid, dental pulp, peripheral blood, bone, cartilage, heart, lung, kidney, liver, brain, spinal cord, skin and tumor tissues.

In some embodiments, human umbilical cord tissues as MSC-containing tissues are used. That is, using hyperthermia in the presence or absence of other disease-mimicking conditions to treat human umbilical cord tissues to produce MSC having enhanced proliferation and therapeutic potencies including anti-inflammation, immunomodulation, secretion of protein, migration, homing, engraftment or differentiation characteristics. Human umbilical cord tissues are collected under sterile conditions from full-term pregnant healthy donors immediately following delivery. An umbilical cord or a section thereof will be transported from a clinical site of the delivery to a processing laboratory in a sterile container containing preservative medium maintained at an appropriate temperature, preferably 2 to 8 degrees Celsius. The umbilical cord tissues, which are free of and distinct from cord blood, comprise amniotic epithelium, two arteries and one vein, and Wharton's Jelly. In several embodiments of this invention, the umbilical cord tissues are used as a bulk for hyperthermia exposure and MSC isolation. That is, no component of the umbilical cord tissues is removed prior to tissue culture.

The culture and/or passaging conditions for MSC-containing tissues and/or MSC according to several embodiments can be practiced using any culture and/or passaging conditions suitable for MSC-containing tissues incubation or MSC expansion as disclosed herein, preferably using DMEM/F12 as the basic growth medium. That is, these embodiments can be practiced using any cell culture or passaging conditions, preferably using DMEM/F12 as the basic growth medium, when combined with hyperthermia in the presence or absence of other disease-mimicking factors as disclosed herein, to produce MSC having enhanced proliferation and therapeutic potencies including anti-inflammation, immunomodulation, secretion of protein, migration, homing, engraftment or differentiation characteristics. As used herein, the phrase “cell culture and/or passaging conditions” includes, but is not limited to, basic growth media, sera, nutrients, additional growth factor supplements, cell confluence rates for passaging, digestive enzymes used for cell detachment and dissociation during passaging, cell seeding densities, the stages of cells (passages) to harvest for cell bank storage or use, and combinations thereof.

In some embodiments, the culture conditions for MSC-containing tissues can be identical to or different from those used for the subsequent MSC that are isolated from the MSC-containing tissues, depending on the purpose of MSC. That is, when using human umbilical cord tissues as the MSC-containing tissues, the culture conditions for human umbilical cord tissues can be identical to or different from those used for human umbilical cord derived MSC.

Stem Cell Banking

In some embodiments, method of generating a bank of stem cells, preferably mesenchymal stem cells (MSC) from human umbilical cord tissues, with enhanced proliferation and therapeutic potencies including anti-inflammation, immunomodulation, secretion of protein, migration, homing, engraftment or differentiation characteristics after exposure of the stem cell-containing tissues, stem cells themselves, or both, to hyperthermia in the presence or absence of other disease-mimicking factors as disclosed herein is provided.

In some embodiments, the MSC from human umbilical cord tissues with enhanced proliferation and therapeutic potencies generated with the methods disclosed herein are banked at an appropriate stage of the cells (passages). Once cultures of stem cells are established, the cultures should be transferred to fresh medium when a sufficient cell density (e.g. 80-90% confluence) is reached. That is, formation of a monolayer of cells, with a cell density regarded as over-confluence (e.g. 100% confluence) should be prevented or minimized. Alternatively, the culture system may be agitated to prevent the cells from sticking or growing in suspension cell culture system.

In some embodiments, the cell banks for stem cells from a specific donor unit include at least one Master Cell Bank and at least one Working Cell Bank. As used herein, a “Master Cell Bank” is a pool of stem cells that have been prepared from a human umbilical cord tissue unit from a single donor. A “Working Cell Bank” is a pool of stem cells derived from a Master Cell Bank.

In one embodiment, the stem cells in cell banks are cryopreserved in the invention. Cryopreservation of cells may be carried out according to any well-known methods, such as those described in Doyle et al., 1995, Cell and Tissue Culture [ref]. For example, but not by way of limitation, cells may be suspended in a “freeze medium” such as, for example, culture medium further comprising 10-90% fetal bovine serum (FBS) and 10% dimethylsulfoxide (DMSO), at a density, for example, of about 1-10×10⁶ cells per milliliter. The cells are dispensed into glass or plastic ampoules that are then sealed and transferred to a freezing chamber of a programmable freezer. The optimal rate of freezing may be determined empirically. For example, a freezing program that gives a change in temperature of about −1° C. per minute through the heat of fusion may be used. Once the ampoules have reached about −180° C., they will be transferred to a liquid nitrogen storage area. Cryopreserved cells can be stored for a period of years and samples of the cryopreserved cells should be checked at an appropriate frequency (e.g. every 6 or 12 months) after the establishment of storage for maintenance of viability and/or potency.

The cryopreserved cells in the cell banks disclosed herein can be “woken up” by thawing and then used to produce working cell banks from master cell banks or to produce stem cell therapeutics from the working cell banks as needed. Thawing should generally be carried out rapidly, for example, by transferring an ampoule from liquid nitrogen to a 37 degree Celsius water bath. The thawed contents of the ampoule should be immediately transferred under sterile conditions to a culture vessel containing an appropriate medium, for example, DMEM/DF12 supplemented with 10% FBS, with or without growth factors, for subculture and expansion as described in this invention.

In some embodiments, the efficiency and scale-up capacity is enhanced for the manufacture of stem cells from a certain stem cell-containing tissue by 1) accelerating the migration of stem cells from the stem cell-containing tissues, decreasing the amount of time that is required for stem cells to leave their sites from their parental tissues and to grow separately in a culture; 2) enhancing the proliferation rate of stem cells in culture, reducing the amount of time to reach a certain stage of cells (passage) or to obtain a desired number of stem cells for cell bank storage or utility.

In some embodiments, the therapeutic potencies of stem cells is enhanced. In such embodiments, a stem cell will gain increased therapeutic potencies by exposing its parental stem cell-containing tissues, themselves, or both to hyperthermia in the presence or absence of other disease-mimicking conditions. As used herein, a stem cell is considered to have increased therapeutic potencies if the stem cell has a measurable increase for the readouts in potency assays, whether in vitro, in vivo, or both, reflecting the in vivo migration, proliferation, survival, engraftment, anti-inflammation, immunomodulation, secretion of factors, and/or differential abilities.

In one embodiment stem cells are provided for (e.g. mesenchymal stem cells (MSC)) for use in a variety of autoimmune diseases and disorders. As used herein, the term “autoimmune diseases and disorders,” refers to a condition that body's immune system attacks healthy cells and impairs the normal functions of the attacked tissues or organs, such as, for example, rheumatoid arthritis where the body's immune system attacks the joints and causes tender, swelling and function impairment of the joints; Crohn's disease where the body's immune system attacks the digestive tract and causes abdominal pain, diarrhea, fever, and weight loss; multiple sclerosis where the body's immune system attacks the oligodendrocytes of the myelin that covers nerve fibers, thus causing neurologic dysfunctions. Other autoimmune diseases and disorders include, but are not limited to, type 1 diabetes, psoriasis, systemic lupus erythematosus (lupus), Addison's disease, graves' disease, Sjögren's syndrome, Hashimoto's thyroiditis, myasthenia gravis, vasculitis, pernicious anemia, celiac disease.

In another embodiment, produces stem cells are produced (e.g. MSC) for use in a variety of liver diseases and disorders. As used herein, the term “liver diseases and disorders,” refers to any conditions that damage the liver and/or prevent it from normal functioning, including but not limited to, cirrhosis where chronic liver damage from a variety of causes leading to scarring and liver failure; alcoholic hepatitis where liver inflammation caused by drinking too much alcohol, for example. Other liver diseases and disorders include, but are not limited to, non-alcoholic fatty liver disease and infectious hepatitis (e.g. hepatitis B).

In another embodiment stem cells are produced (e.g. MSC) for use as a drug delivery platform to treat a variety of malignant diseases. As used herein, the terms “malignant diseases”, “cancer” or “tumor” refer to any diseases in which abnormal cells divide uncontrollably and destroy body tissues. The malignant diseases include, but are not limited to, breast cancer, colon cancer, lung cancer, liver cancer, brain cancer and gastric cancer.

In some embodiments, the compositions of stem cell as therapeutics are formulated by suspending an appropriate amount of cells in a pharmaceutically acceptable carrier solution. The final concentration of the stem cell in the solution can be 1×10⁵ per milliliter, 2×10⁵ per milliliter, 5×10⁵ per milliliter, 1×10⁶ per milliliter, 1.5×10⁶ per milliliter, 2×10⁶ per milliliter, 3×10⁶ per milliliter, 4×10⁶ per milliliter or 5×10⁶ per milliliter. In other aspects, the final concentration of the stem cells in the solution can be any in the range of 1×10⁵ per milliliter to 1×10⁷ per milliliter, depending on the specific purpose of a therapeutic. As used herein the phrase “pharmaceutically acceptable” means the carrier solution does not cause any adverse reactions, or acceptable adverse reactions, if any, when administered, either systemically or locally, to a human or other mammal. Such carriers are non-toxic, and do not create an inflammatory or anergic response in the body, and have been used in human or other mammals with an excellent safety profile. Pharmaceutically acceptable carrier solutions for practicing the invention include any of the well-known components useful for immunization such as, for example, culture media and phosphate buffered saline. Additional physiologically acceptable carriers and their formulations are well-known and generally described in, for example, Remington's Pharmaceutical Science (18th Ed., Gennaro, Mack Publishing Co., Easton, Pa., 1990) and the Handbook of Pharmaceutical Excipients (4th Ed., Rowe et al. Pharmaceutical Press, Washington, D.C.), [refs] each of which is incorporated by reference.

In some embodiments, the therapeutics of the stem cell in this invention is administered to the subject systemically or locally. As used herein, the term “systemically”, for administration is intended to deliver the stem cell therapeutics into the circulatory system so that the entire body will be affected. Common routes for systemic administration include, but are not limited to, intravenous infusion, intra-arterial infusion, portal vein injection, intraperitoneal injection and intranasal injection. The term “locally”, for administration is intended to deliver the stem cell solution to specific site or sites of the body other than the circulatory system. Common routes for local administration include, but are not limited to, intrathecal injection, intra-articular injection, intra-cerebral injection and any routes of injection directly into a tissue or organ parenchyma.

### EXAMPLES

### Example 1: Study on MSC Isolation Process Using Hyperthermic Conditions

### Experimental Design:

TABLE 1
Grouping

| | Temper- | Temper- | Temper- | Temper- | Temper- |
|---|---|---|---|---|---|
| Culture | ature/ | ature/ | ature/ | ature/ | ature/ |
| medium | Duration | Duration | Duration | Duration | Duration |
| D/F12 + | 37° C. | 38° C., | 38° C., | 39° C., | 39° C., |
| 10% FBS* | | 30 min | 1 hr | 30 min | 1 hr |
| D/F12 + | 37° C. | 38° C., | 38° C., | 39° C., | 39° C., |
| 5% | | 30 min | 1 hr | 30 min | 1 hr |
| UltraGRO | | | | | |
| (UG) | | | | | |

*1. “D/F12 + 10% FBS” is abbreviated to “FBS system” and D/F12 + 5% UltraGRO is abbreviated to “UG system”; 2. Only for the 6-hour incubation after attachment, FBS was adjusted to 20% and UG was adjusted to 10%.
MSC isolation under hyperthermic conditions.

The umbilical cord tissues of each group was cut and attached, and incubated at 37-39° C. for 30 min or 1 hr respectively. After incubation, the umbilical cord tissues were incubated at 37° C. for 6 hours, and then the complete culture medium (i.e., FBS or UG system in Table X) was added. The cells were then cultured for about 10 days. The cells were then trypsinized and counted. The statistics of each group are as follows:

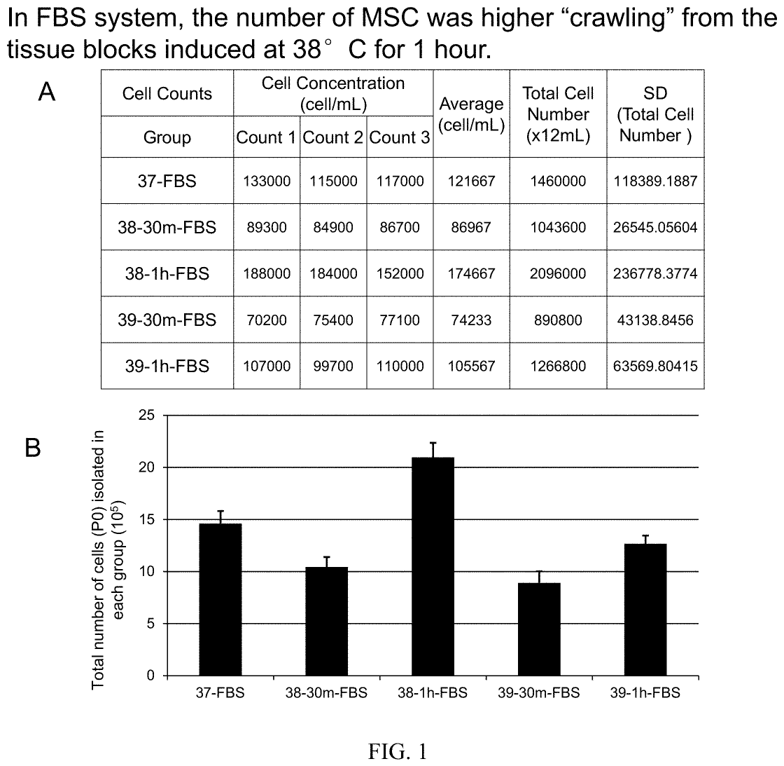

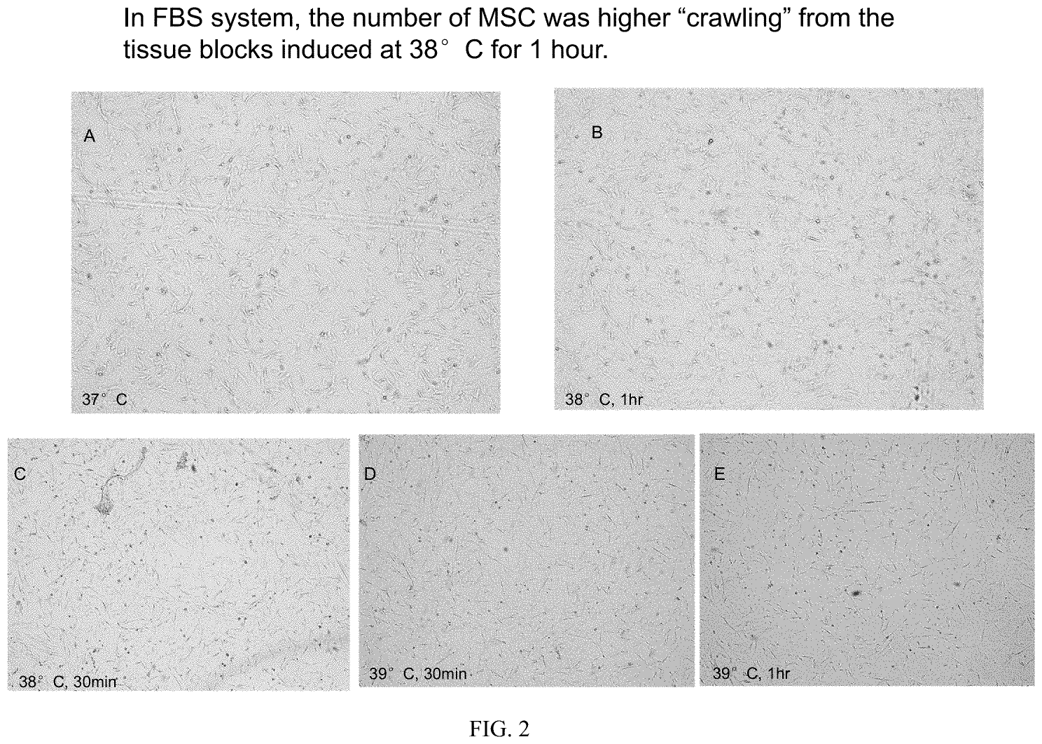

In FBS system, the number of MSC was higher “crawling” from the tissue blocks induced at 38° C. for 1 hour. Each block on average produced about 20% more MSC than that in the 37° C. group (FIGS. 1 and 2);

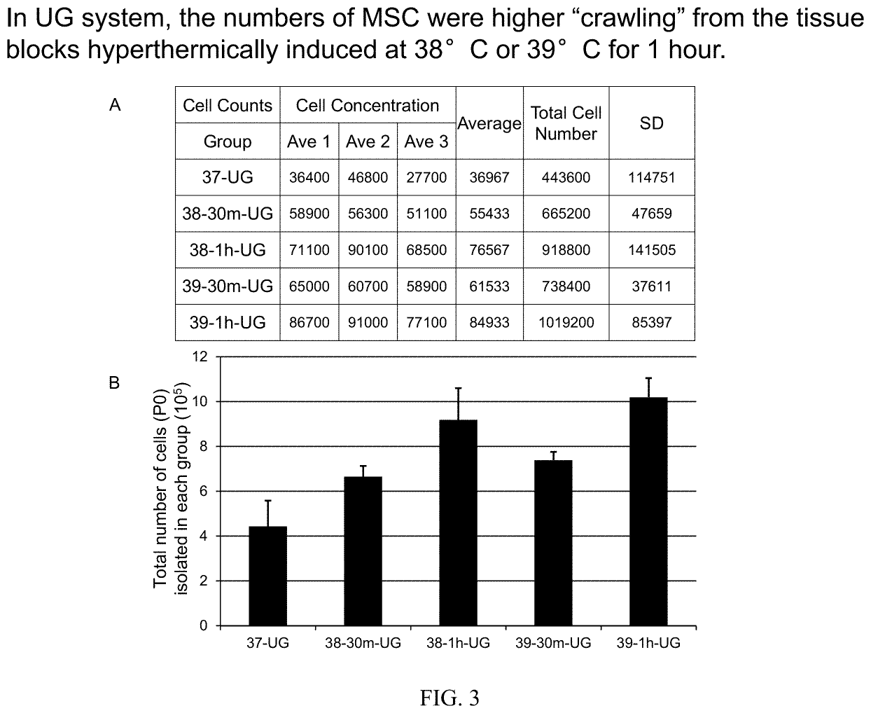

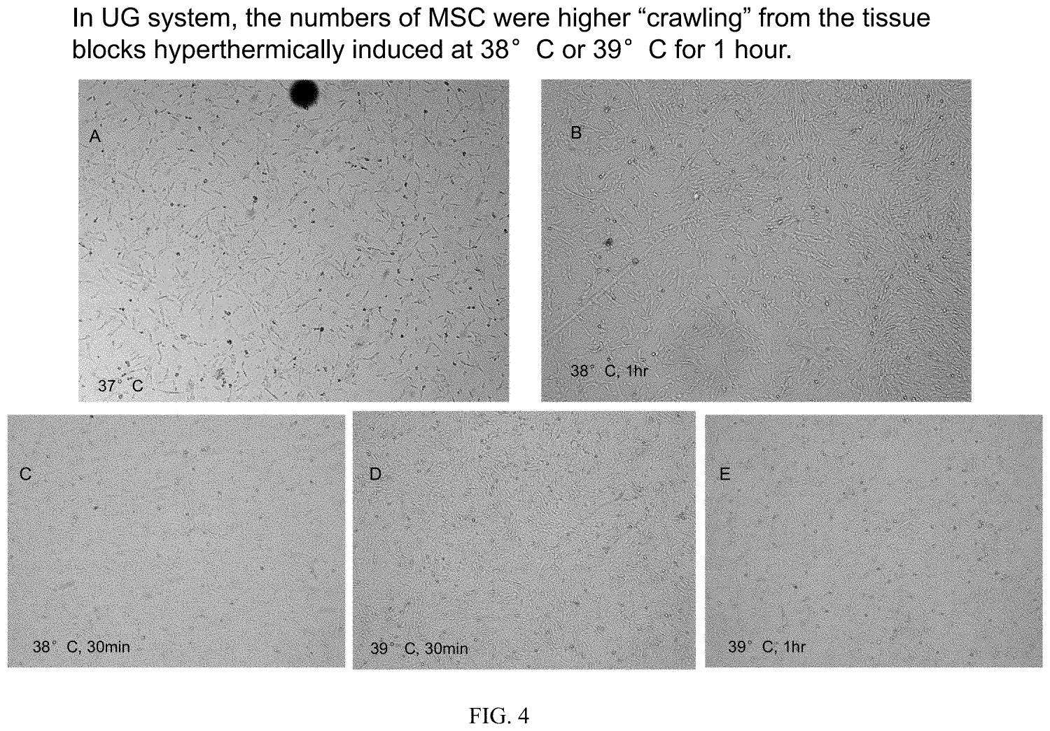

In UG system, the numbers of MSC were higher “crawling” from the tissue blocks hyperthermically induced at 38° C. or 39° C. for 1 hour. Each block produced 40-80% more MSC than that in the 37° C. group (FIGS. 3 and 4).

The results of this experiment indicate: the isolation efficiencies of hyperthermic groups is higher than that of 37° C. group; the MSC isolation efficiencies of 38° C. groups are higher than those of 39° C. groups; and in each system, the hyperthemic 1 h groups were more efficient than the 30 min groups.

### Example 2: Study on the Expansion of MSC Isolated Using Hyperthermic Conditions

Result:

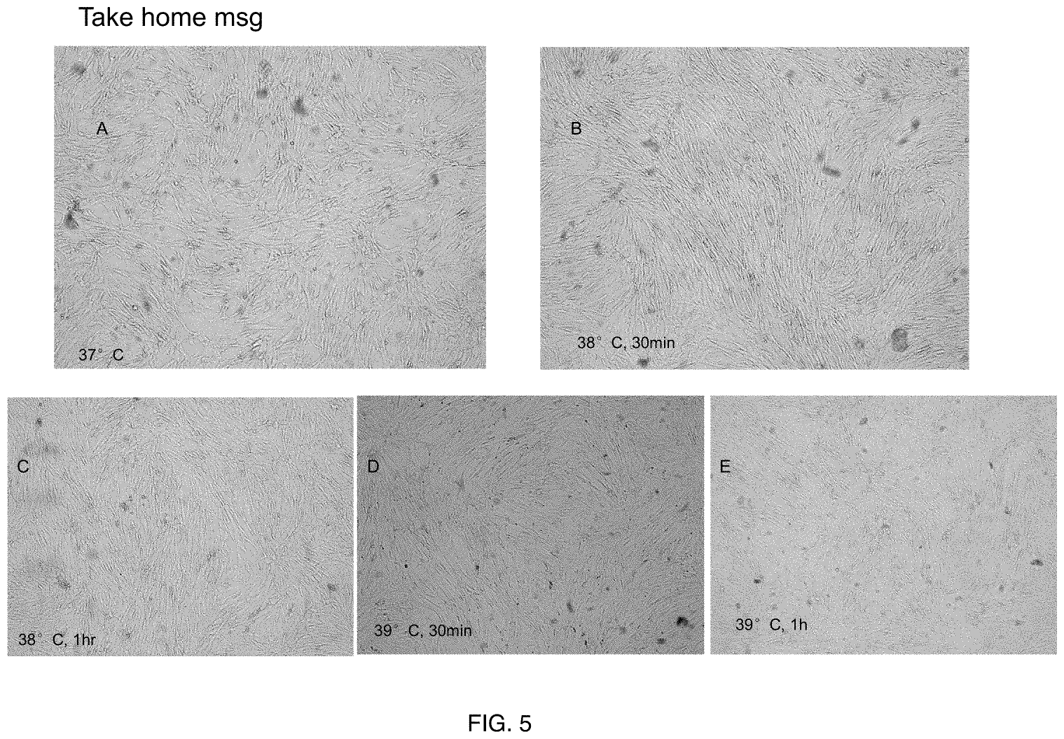

The cells induced at different temperatures were expanded to P6 in corresponding culture media. The subcellular state, cell number and cell diameter of each passage were statistically analyzed during expansion. (FIG. 5)

In FBS system, the cell morphology was not significantly different between passages within (including) P6. The cell numbers increased gradually along with the passage of cells. The total number of cells induced hyperthermically at 38° C. for 1 hour was significantly higher than those of other groups. Cell diameter increased slowly along with passage, the difference between groups was not significant.

In UG system, the cell morphology in each group significantly increased after (including) P4. The number of cells gradually increased along with the passage. The numbers of cells induced hyperthermically at 38° C. and 39° C. for 1 hour were significantly higher than those of other groups. Cell diameter after (including) P3 was significantly increased, and diameters of cells from 39° C. groups were significantly higher than those of other groups.

2.1 Cell Morphology

In FBS system: Cells grew well and were spindle-like. Before (including) P3, the difference of morphology between groups was not visible and cells induced at 39° C. after (including) P4 were larger by naked eyes than those from other groups.

In UG system: Cells in each group before (including) P3 grew well with relatively small size and with no visible difference between groups. But after (including) P4 the cell size was increased by naked eyes.

2.2 Cell Numbers in Each Passage

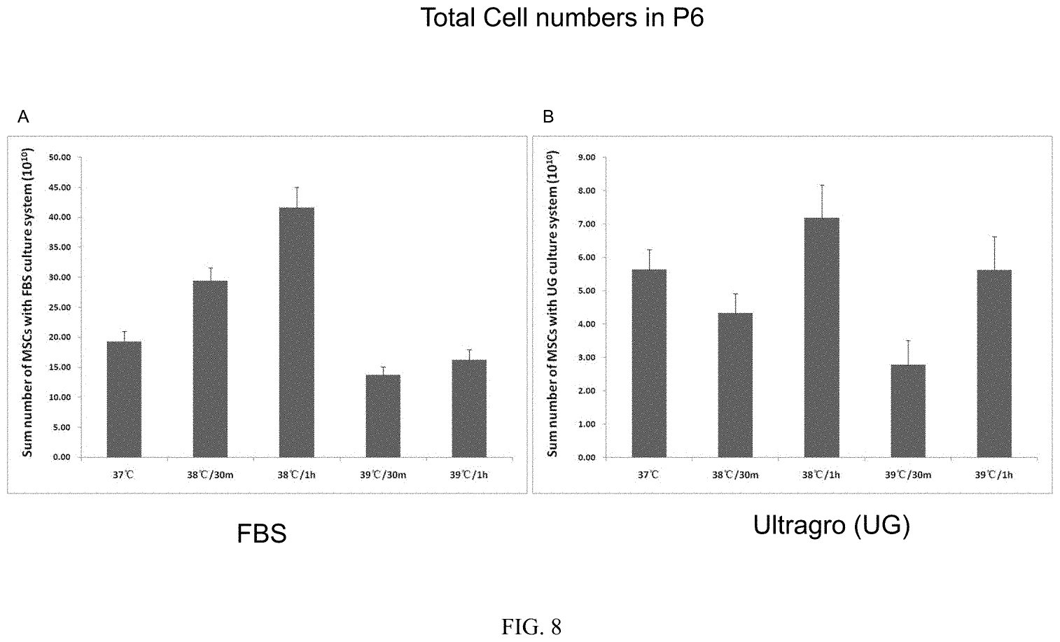

The total number of cells in FBS system increased rapidly along with passaging. The total number of cells in 1-hour hyperthermically induced group was significantly higher than those in other groups. The cell numbers of the group of 38° C./1 hr after (including) P3 were significantly higher than those in 39° C. groups. The total cell number of the group of 38° C./1 hr in P6 was significantly higher than those in other groups, which e.g., is 2.15 folds higher than the group of 37° C. (FIGS. 6 and 8)

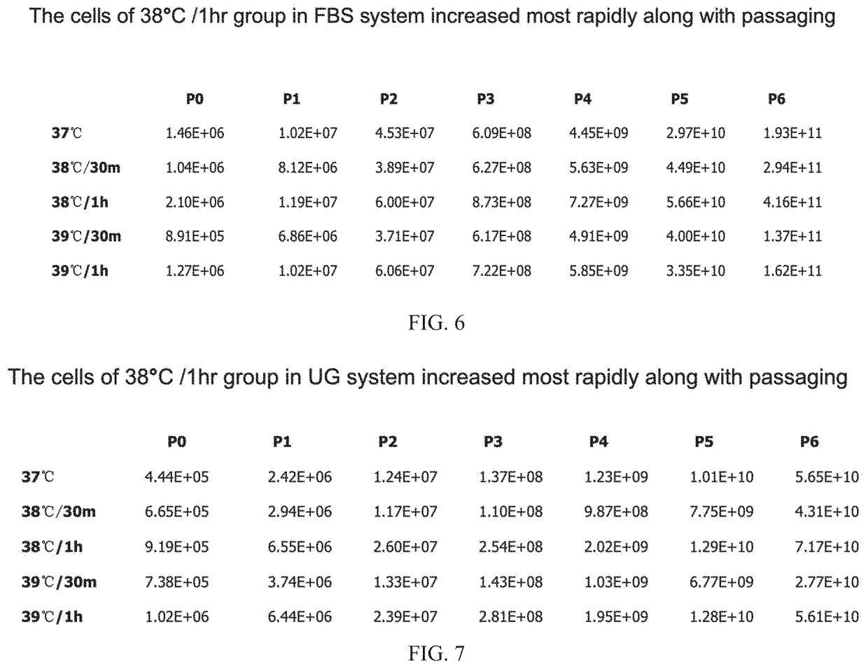

The total number of cells in UG system was lower than that in FBS system at each passage. The total cell numbers in the groups of 1-hour hyperthemically induction were significantly higher than those in other groups. The total number of cells in the group of 38° C./1 hr in P6 was the highest, which e.g., is 1.28 folds higher than the group of 37° C. (FIGS. 7 and 8)

2.3 Cell Diameters in Each Passage

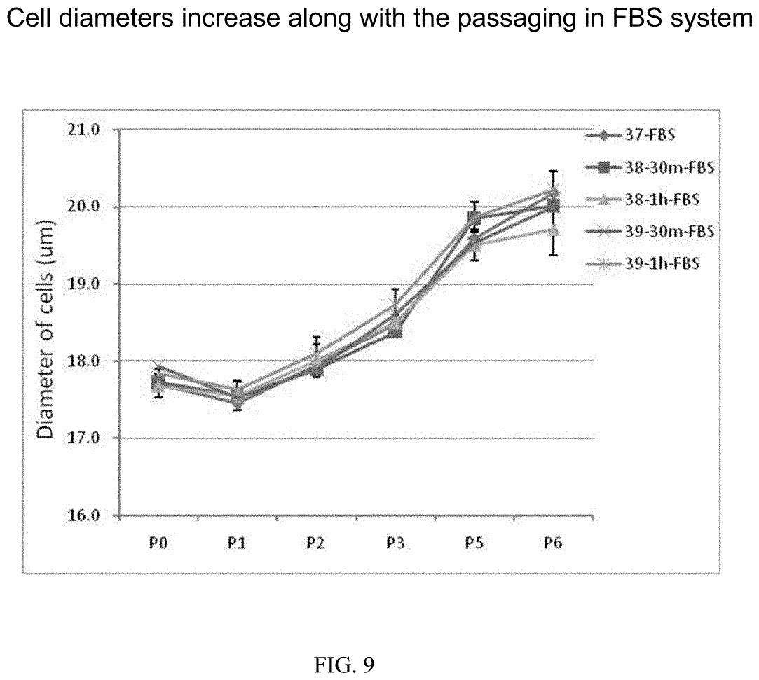

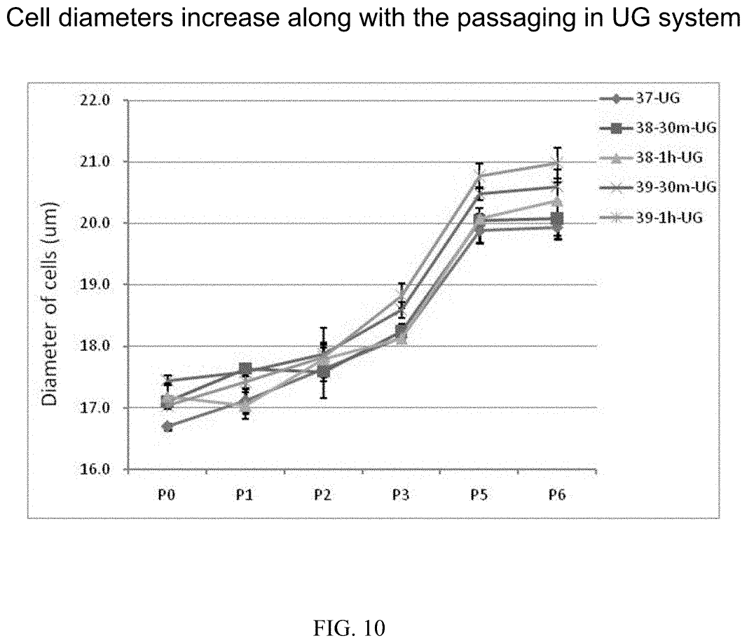

The cell diameters of each passage were measured. Along with passaging, cell diameters continued to increase. In FBS system cell diameters changed more slowly and there was no significant difference between groups. However in UG system, the differences between the cells were greater. After (including) P3, the cell diameters in the groups of 39° C./30 min and 39° C./1 hr were significantly larger than those of other groups. (FIGS. 9 and 10)

### DISCUSSION

1) Morphology and numbers of cells in each group after continuous culture:

The final yield of FBS system was significantly higher than that of UG system, among which 38° C./1 hr group in FBS system had the highest efficiency.

2) The cell diameter of 38° C./30 min group in FBS system was more stable and the difference between groups was not significant. The cell diameter of 39° C. groups in UG system was significantly higher than that of other groups after (including) P3.

### Example 3: Study on the Functions of MSC (P6) Isolated Using Hyperthermic Conditions

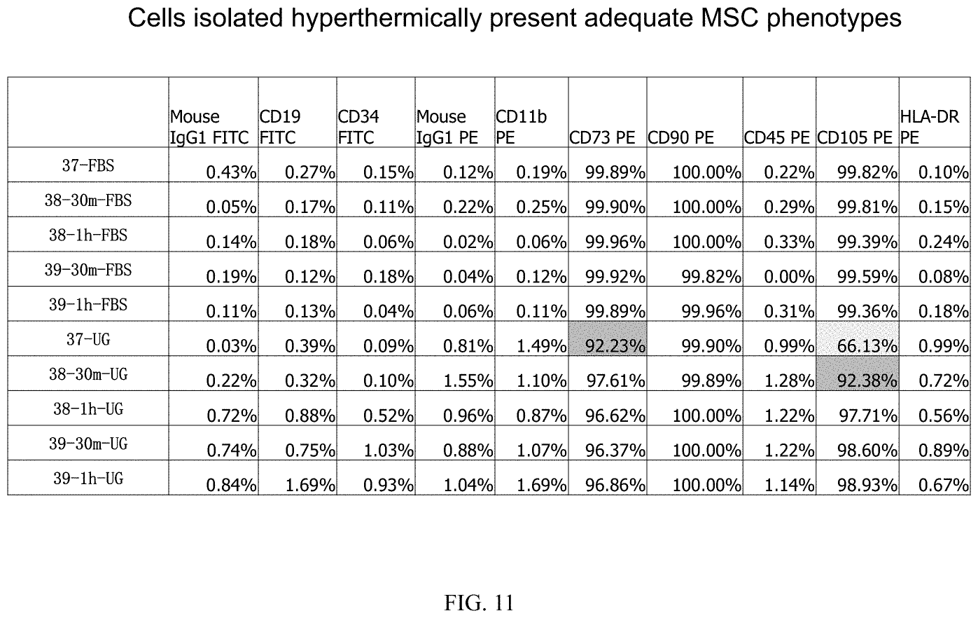

3.1 Phenotyping assay using flow cytometry (FIG. 11)

Negative: CD19, CD34, CD11b, CD45 and HLA-DR

Positive: CD73, CD90 and CD105

3.2 CCK-8 cell proliferation assay

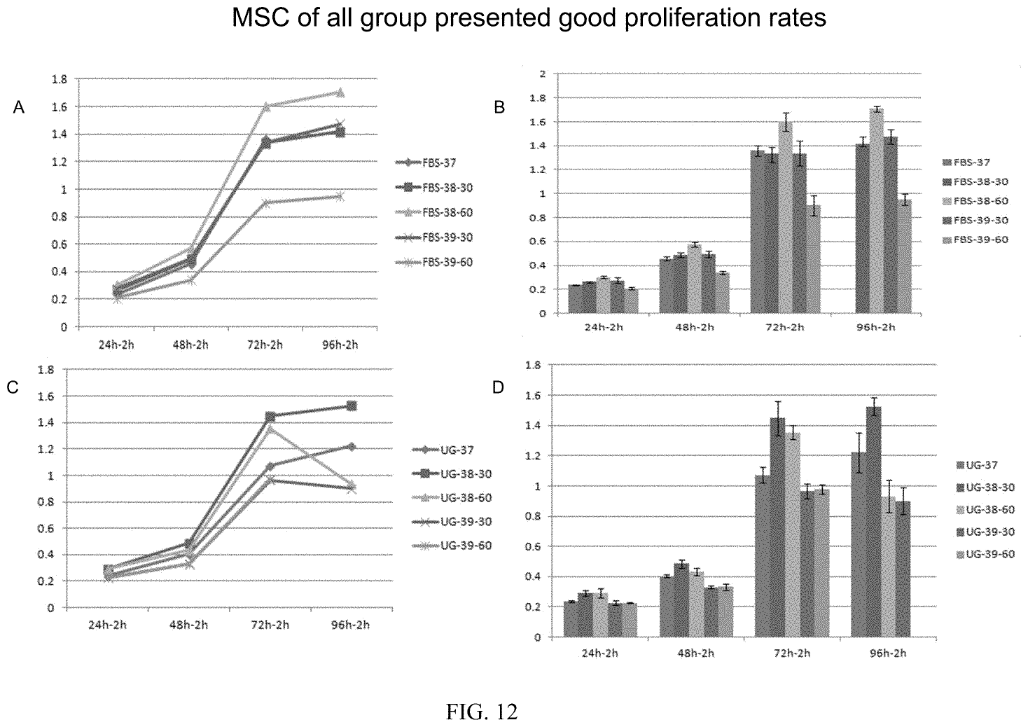

MSC of all group presented good proliferation rates. In FBS system, MSC of the 38° C./1 hr group have the highest proliferation rate and in UG system MSC of the 38° C./30 min group have the highest proliferation rate. (FIG. 12)

3.3 β-Gal Senescence Assay

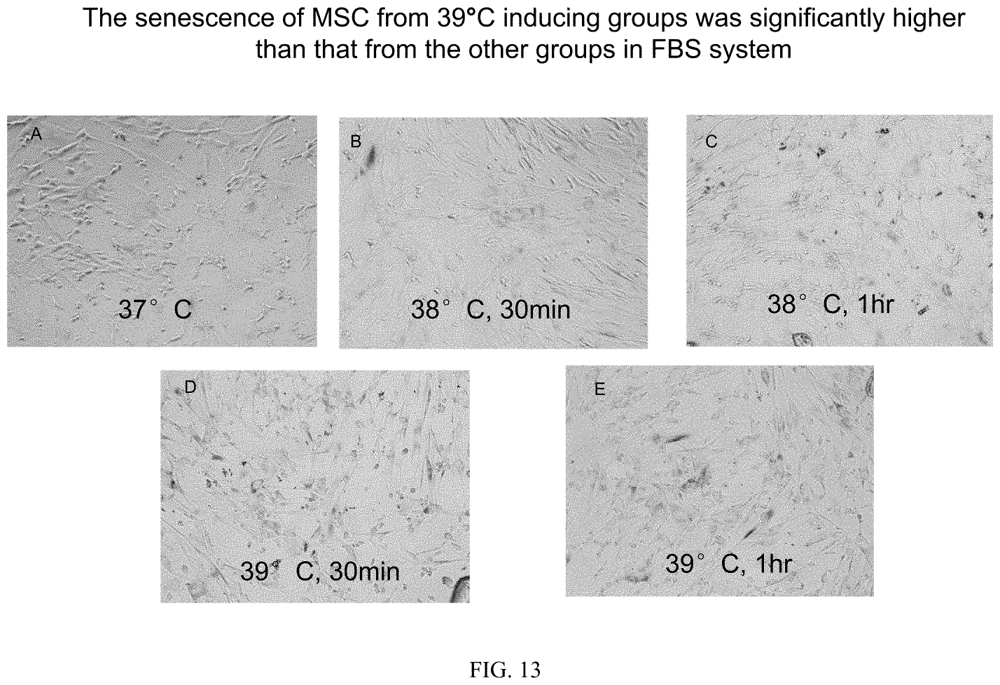

In FBS system, SA-β-gal staining showed that the senescence of MSC from 39° C. inducing groups was significantly higher than that from the other groups (FIG. 13).

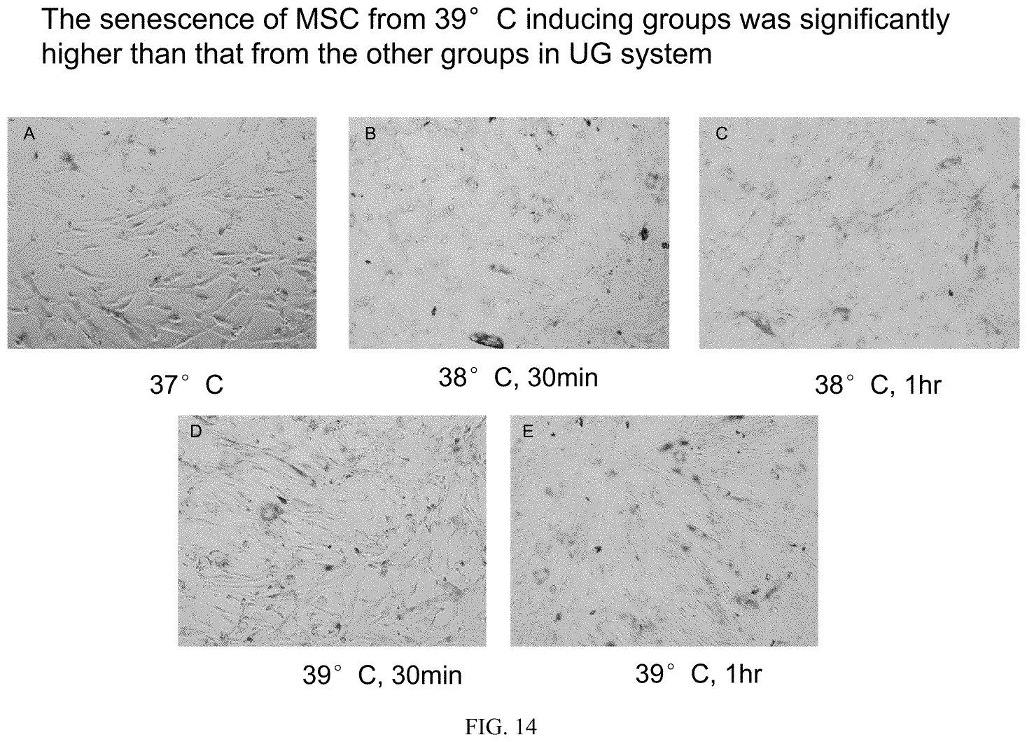

In UG system, SA-β-gal staining showed that the senescence of MSC from 39° C. inducing groups was significantly higher than that from the other groups (FIG. 14).

3.4 TNF-α Immunomodulatory Inhibition Assay

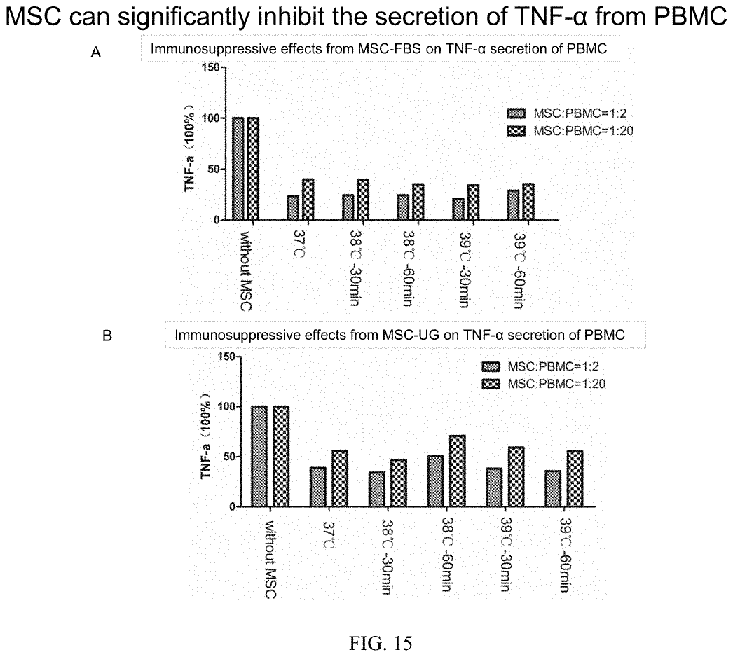

After co-culture of MSC and PHA-PBMC for 72 hours, the concentration of TNF-α in the supernatant was tested. The results indicate that the stimulated PBMC can secrete TNF-α and co-culture with MSC can significantly inhibit the secretion of TNF-α. The inhibitory effect was positively correlated with cell number of MSC. There was no significant difference in the immunosuppressive effects from MSC between groups. (FIG. 15)

### DISCUSSION

1) The results of CCK-8 proliferation assay showed that the cells in 38° C. groups were stable and the proliferation rates were significantly higher than those in other groups. The cell proliferation rates in 39° C. groups were significantly lower than those in other groups.

2) Results of flow cytometric assay showed that the cells subcultured up to P6 in each system presented the MSC phenotype.

3) The results of β-gal cell senescence test showed that the cell senescence in the 39° C. groups was higher than that in the 38° C. and 37° C. groups, but there was no significant difference in the senescence between the 38° C. groups and the 37° C. group. There was no significant difference between the cells induced for 30 min and 1 hr.

4) The results of TNF-α immunosuppression assay showed that MSC of both systems had immunosuppressive effects, and the immunosuppressive effect was stronger when the concentration of MSC was higher.

The term “comprising” as used herein is synonymous with “including,” “containing,” or “characterized by,” and is inclusive or open-ended and does not exclude additional, unrecited elements or method steps.

While this invention focuses on MSC, it is understood that using hyperthermic conditions can be potentially used to enhance the proliferation and therapeutic functions of any types of stem cells. Therefore, the utility of hyperthermic conditions to treat any types of stem cells or any of their origin tissues, to produce stem cells with enhanced proliferation and therapeutic functions, is within the scope of this invention.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of treating mesenchymal stem cells (MSC)-containing tissues with hyperthermic conditions for the isolation of the MSC with enhanced proliferation rate and therapeutic potencies compared to control MSC isolated without hyperthermic conditions comprising:
(a) obtaining an MSC-containing tissue;
(b) culturing the MSC containing tissue with an optimized medium;
(c) exposing the MSC containing tissue to hyperthermic condition; and
(d) providing a therapeutic composition to treat a medical disease.

2. The method of claim 1, wherein the hyperthermic conditions produce MSC-containing tissue with enhanced proliferation rate and therapeutic potencies when compared to a control.

3. The method of claim 1, wherein the hyperthermic conditions comprise temperatures between about 37 to about 40 degrees Celsius (° C.).

4. The method of claim 1, wherein the optimized medium includes one or more supplements selected from growth factors (bFGF, EGF) and hormones (Dexamethasone), antibiotics (Pen/Strap, Amphotericin B), serum, and xeno-free serum replacement.

5. The method of claim 1, wherein the MSC-containing tissues comprises: human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

6. The method of claim 5, wherein the human umbilical cord tissues is disaggregated by mechanical cutting the tissue without enzymatic digestion before culturing in the optimized medium.

7. The method of claim 5, wherein the human umbilical cord tissues are mechanically cut into small sections comprising 1-1.5 mm in diameter and not washed in order to preserve the environment of the tissues after mechanical injury by cutting or agitation and then directly used for culturing.

8. The method according to claim 1, wherein obtaining MSC-containing tissues comprises tissue taken from bone marrow, adipose tissues, blood, amniotic fluid, dental pulp and placenta.

9. A method according to claim 1, wherein resulting MSC with enhanced proliferation rate and therapeutic potencies are suitable use in a therapeutic composition to treat a medical disease includes: anti-inflammation, immunomodulation, secretion of protein, migration differentiation of the MSC.

10. A method according to claim 1, wherein the hyperthermic conditions may include one or more of the followings in addition to hyperthermia treatment: mechanical cutting or agitation; damage associated molecular patterns (DAMPs) such as HGMB1, S100B; pathogen associated molecular patterns (PAMPs) such as peptidoglycan (PGN), and double-stranded RNA; heat shock proteins; inflammatory cytokine (such as TNF-α, IFN-γ, interleukin-1 (IL-1) etc.) stimulation; stem cell migration related proteins (CXCL12, G-CSF, SCF, PDGF-BB, etc.) stimulation and hypoxia (<20% O₂).

11. The method of claim 10, wherein the damage associated molecular patterns (DAMPs) include, but are not limited to, HGMB1 (the chromatin-associated protein high-mobility group box 1), S100B, Purine metabolites (ATP, adenosine, and uric acid).

12. The method of claim 10, wherein the pathogen associated molecular patterns (PAMPs) include, but are not limited to, peptidoglycan (PGN), and double-stranded RNA.

13. The method of claim 10, wherein heat shock proteins include, but are not limited to, HSP 40, HSP 60, HSP 70, HSP 72, HSP 78, HSP 90, HSP 104, HSP 110.

14. The method of claim 10, wherein the inflammatory cytokines include, but are not limited to, TNF-α, IFN-γ, TGF-β, IL-1, IL-6, IL-8.

15. The method of claim 10, wherein the stem cell migration related proteins include, but are not limited to, CXCL12 (stromal cell-derived factor-1: SDF-1), CCL2 (monocyte chemotactic protein-1: MCP-1), CCL5 (regulated on activation, normal T cell expressed and secreted: RANTES), CCL8, G-CSF, SCF, PDGF-BB.

16. A method according to claim 1, wherein the providing a therapeutic composition to treat a medical disease, includes a composition to treat autoimmune diseases or liver diseases, or cancer.

17. The methods of claim 16, wherein autoimmune diseases comprise Crohns' diseases, ulcerative colitis, multiple sclerosis, rheumatoid arthritis, systemic lupus erythematosus, autoimmune pancreatitis, Type 1 diabetes, systemic sclerosis.

18. The methods of claim 16, wherein liver diseases comprise liver failure, cirrhosis, hepatic steatosis, hepatitis.
